(19) United States
(12) Patent Application Publication
(10) Pub. No.: US 2008/0319327 A1
(43) Pub. Date: Dec. 25, 2008

(54) BODY-WORN SENSOR FEATURING A LOW-POWER PROCESSOR AND MULTI-SENSOR ARRAY FOR MEASURING BLOOD PRESSURE

(75) Inventors: Matthew J. Banet, Del Mar, CA (US); Zhou Zhou, San Diego, CA (US); Kenneth Robert Hunt, Vista, CA (US); Henk Visser, II, San Diego, CA (US)

Correspondence Address: WilmerHale/Triage Wireless, 60 State Street, Boston, MA, 02109, US

(73) Assignee: Triage Wireless, Inc., San Diego, CA (US)

(21) Appl. No.: 12/146,432

(22) Filed: Jun. 25, 2008

Related U.S. Application Data
(60) Provisional application No. 60/946,036, filed on Jun. 25, 2007.

(51) Int. Cl. A61B 5/021

(52) U.S. Cl. 600/485

(57) ABSTRACT

A system is described that continuously measures a patient's blood pressure over a length of time. The system features a sensor assembly featuring a flexible cable configured to wrap around a portion of a patient's arm. The flexible cable features a back surface that includes at least two electrodes that are positioned to contact the patient's skin to generate electrical signals. It additionally features an optical sensor that includes at least one light source and at least one photodetector. These components form an optical sensor that is configured to generate an optical signal by detecting optical radiation emitted by the at least one light source and reflected from a blood vessel underneath the patient's skin.

### CROSS REFERENCES TO RELATED APPLICATION

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to medical devices for monitoring vital signs, e.g., blood pressure.

2. Description of the Related Art

Pulse transit time (PTT), defined as the transit time for a pressure pulse launched by a heartbeat in a patient's arterial system, has been shown in a number of studies to correlate to both systolic and diastolic blood pressures. In these studies, PTT is typically measured with a conventional vital signs monitor that includes separate modules to determine both an electrocardiogram (ECG) and pulse oximetry. During a PTT measurement, multiple electrodes typically attach to a patient's chest to determine a time-dependent electrical waveform, i.e. an ECG, than includes a sharp spike called the ‘QRS complex’. This feature indicates an initial depolarization of ventricles within the heart and, informally, marks the beginning of the heartbeat and pressure pulse that follows. Pulse oximetry is typically measured with a bandage or clothespin-shaped sensor that attaches to a patient's finger. A typical pulse oximeter sensor includes optical systems operating in both the red and infrared spectral regions. A photodetector measures radiation emitted from the optical systems and transmitted through the patient's finger. Other body sites, e.g., the ear, forehead, and nose, can also be used in place of the finger. During a measurement, a microprocessor analyses both red and infrared radiation measured by the photodetector to determine the patient's blood oxygen saturation level and a time-dependent optical waveform, i.e. a photoplethysmograph (PPG). Time-dependent features of the optical waveform indicate both pulse rate and a volumetric absorbance change in an underlying artery (e.g., in the finger) caused by the propagating pressure pulse.

Typical PTT measurements determine the time separating a maximum point on the QRS complex (indicating the peak of ventricular depolarization) and a foot of the optical waveform (indicating the beginning of the pressure pulse). PTT depends primarily on arterial compliance, the propagation distance of the pressure pulse (closely approximated by the patient's arm length), and blood pressure. To account for patient-dependent properties, such as arterial compliance, PTT-based measurements of blood pressure are typically ‘calibrated’ using a conventional blood pressure cuff. Typically during the calibration process the blood pressure cuff is applied to the patient, used to make one or more blood pressure measurements, and then removed. Going forward, the calibration blood pressure measurements are used, along with a change in PTT, to determine the patient's blood pressure and blood pressure variability. PTT typically relates inversely to blood pressure, i.e., a decrease in PTT indicates an increase in blood pressure.

A number of issued U.S. patents describe the relationship between PTT and blood pressure. For example, U.S. Pat. Nos. 5,316,008; 5,857,975; 5,865,755; and 5,649,543 each describe an apparatus that includes conventional sensors that measure an ECG and optical waveform, which are then processed to determine PTT.

Studies have also shown that a property called vascular transit time (‘VTT’), defined as the time separating two plethysmographs measured from different locations on a patient, can correlate to blood pressure. Alternatively, VTT can be determined from the time separating other time-dependent signals measured from a patient, such as those measured with acoustic or pressure sensors. A study that investigates the correlation between VTT and blood pressure is described, for example, in ‘Evaluation of blood pressure changes using vascular transit time’, Physiol. Meas. 27, 685-694 (2006). U.S. Pat. Nos. 6,511,436; 6,599,251; and 6,723,054 each describe an apparatus that includes a pair of optical or pressure sensors, each sensitive to a propagating pressure pulse, that measure VTT. As described in these patents, a microprocessor associated with the apparatus processes the VTT value to estimate blood pressure.

Other efforts have attempted to use a calibration along with other properties of the plethysmograph to measure blood pressure. For example, U.S. Pat. No. 6,616,613 describes a technique wherein a second derivative is taken from a plethysmograph measured from the patient's ear or finger. Properties from the second derivative are then extracted and used with calibration information to estimate the patient's blood pressure. In a related study, described in ‘Assessment of Vasoactive Agents and Vascular Aging by the Second Derivative of Photoplethysmogram Waveform’, Hypertension. 32, 365-370 (1998), the second derivative of the plethysmograph is analyzed to estimate the patient's ‘vascular age’ which is related to the patient's biological age and vascular properties.

### SUMMARY OF THE INVENTION

This system described herein provides a lightweight, low-power, body-worn sensor that includes a flexible cable that supports a multi-sensor optical array and electrodes. These sensors measure, respectively, optical and electrical waveforms, that are then processed to make a cuffless measurement of blood pressure using PTT. The body-worn sensor may be worn for days or months and operates using AA batteries. The patient may comfortably wear the body-worn sensor throughout the day while participating in their daily activities. The body-worn sensor uses wireless communication to transmit information to a personal computer or display device.

Once measured, the PTT value may be corrected by a property, referred to herein as a ‘vascular index’ (‘VI’), that accounts for the patient's arterial properties (e.g., stiffness and size). VI is typically determined by the shape of the optical waveform, which is measured from the brachial, finger, radial, or ulnar arteries. To accurately measure VI, the optical waveform must be characterized by a high signal strength and signal-to-noise ratio.

In one aspect, the system continuously measures a patient's blood pressure over time and features a sensor assembly featuring a flexible cable configured to wrap around a portion of a patient's arm. The flexible cable features a back surface that includes at least two electrodes that are positioned to contact the patient's skin to generate electrical signals. It additionally features an optical sensor that includes at least one light source and at least one photodetector. These components form an optical sensor that is configured to generate an optical signal by detecting optical radiation emitted by the light source and reflected from a blood vessel underneath the patient's skin.

The system further includes a controller configured to be worn on the patient's body that connects to the sensor assembly through a connector. The controller includes an analog-signal processing circuit featuring a first amplifier configured to receive the electrical signals from the electrodes to generate an analog electrical waveform, and a second amplifier configured to receive the optical signal from the photodetector to generate an analog optical waveform. The controller additionally includes an analog-to-digital converter configured to generate digital optical and electrical waveforms, and a central processing circuit configured to receive the digital electrical and optical waveforms and determine a PTT. A power-regulating circuit in the controller manages power supplied to the analog-signal processing circuit and central processing circuit.

In embodiments the flexible cable features a rectangular cross section. It typically includes a polymer base with conductive traces and sets of metal pads for mounting the light source and photodetector (using, e.g., metal solder). The flexible cable can include connectors that mate to a matched connector comprised by a disposable electrode. Alternatively the electrode is adhered directly to the flexible cable with an adhesive.

In other embodiments the flexible cable includes a first connector in electrical contact with the at least two electrodes, the light source, and the photodetector. In this case the controller includes a second connector configured to mate with the first connector, wherein the second connector is in electrical contact with the analog-signal processing circuit.

Typically the light source or array of light sources mounted on the cable emits radiation near b 570 nm. In other embodiments the controller includes a short-range wireless transceiver configured to transmit information to a remote receiver.

The invention has a number of advantages. In general, the body-worn sensor described features a flexible, comfortable interface to the patient that measures optical and electrical signals. These signals are processed to determine both PTT and VI, which can them be used to make a cuffless, continuous measurement of blood pressure. This simplifies the process of measuring blood pressure, particularly continuous blood pressure in a hospital setting. Ultimately this results in an easy-to-use, flexible system that performs one-time, continuous, and ambulatory measurements. Measurements can be made throughout the day with little or no inconvenience to the caregiver or patient.

These and other advantages are described in detail in the following description, and in the claims.

### BRIEF DESCRIPTION OF THE DRAWINGS

FIG. 1 is a schematic view of body-worn sensor featuring a low-power processing module, multi-sensor array, electrodes, and a pulse oximetry circuit;

FIGS. 2A and 2B are schematic views of, respectively, the body-worn sensor by itself and worn on a patient;

FIGS. 3A and 3B are, respectively, schematic front and side views of three circuit boards housed within a processing module of the body-worn sensor of FIGS. 1, 2A, and 2B;

FIG. 4 is a schematic diagram of the electrical components of the processing module of FIGS. 3A and 3B; and

FIGS. 5A and 5B are schematic views of the body-worn sensor system attached to a patient's arm and wirelessly connected to, respectively, a personal computer and a hand-held bedside monitor.

### DETAILED DESCRIPTION OF THE INVENTION

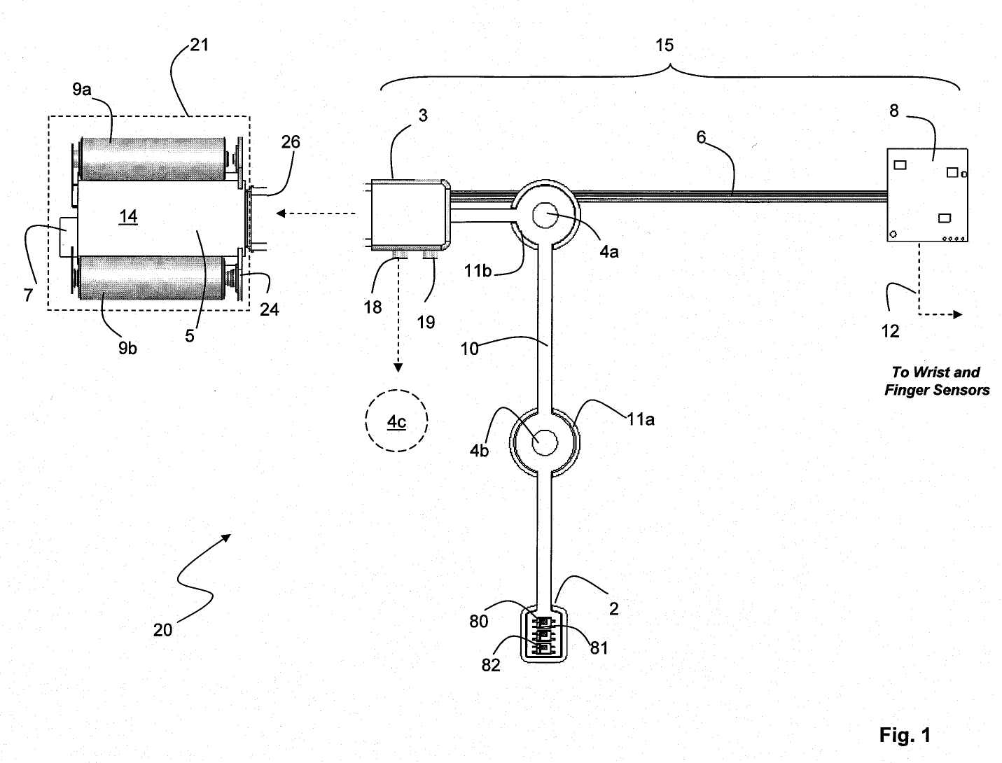

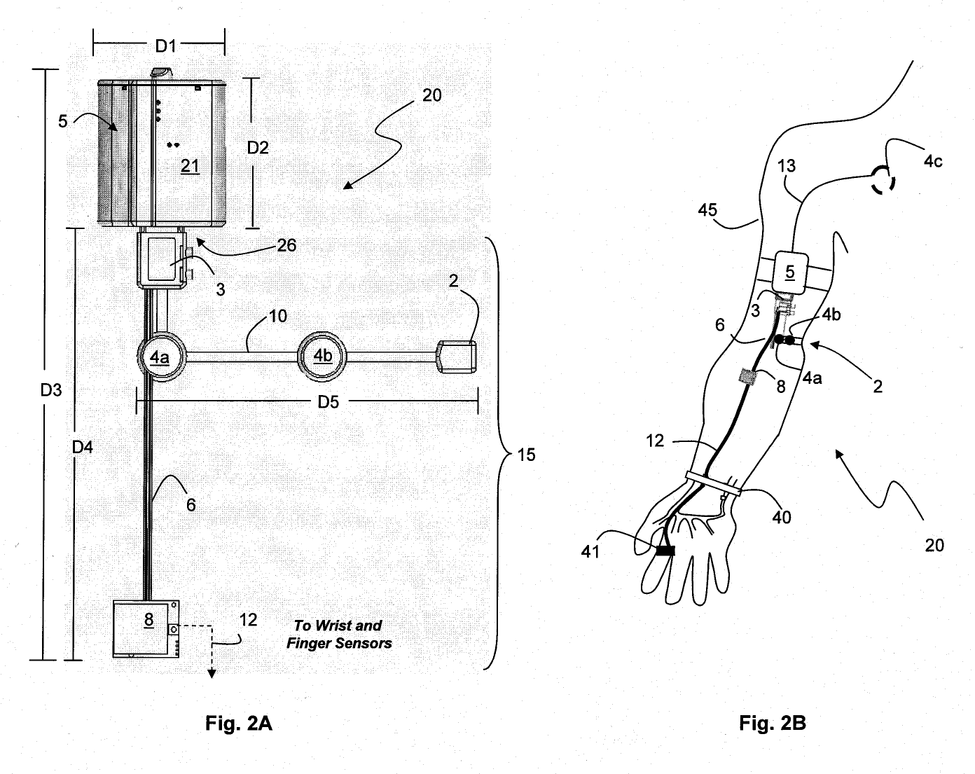

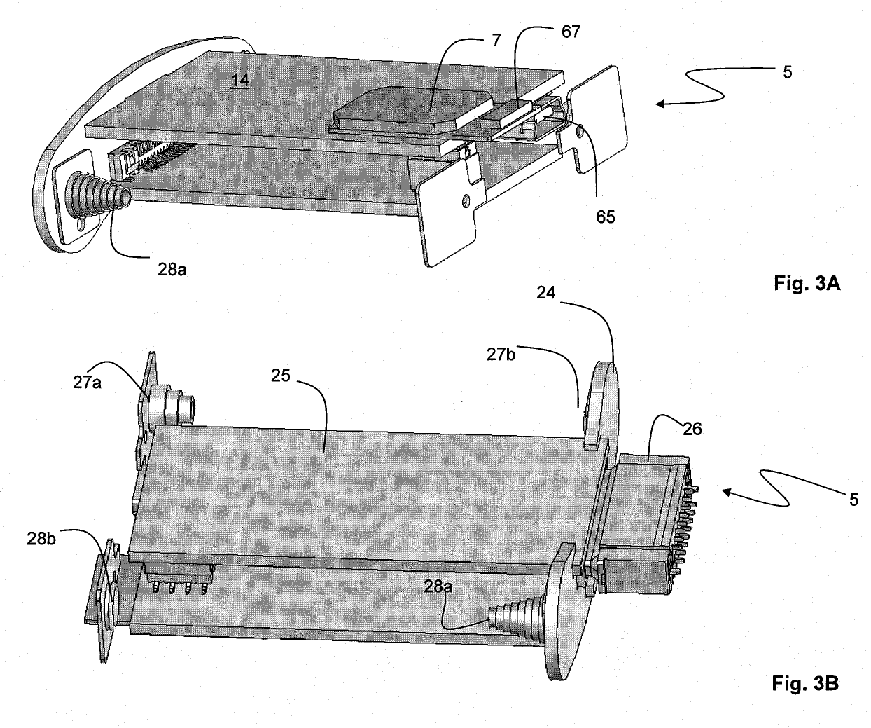

FIGS. 1, 2A, and 2B show a body-worn sensor system 20, according to the invention, featuring a lightweight, low-power processing module 5 connected to a flexible sensor assembly 15 for measuring blood pressure. The body-worn sensor system 20 includes three separate small circuit boards (shown in more detail in FIGS. 3A, 3B) within the processing module 5, all of which are contained within a plastic housing 21. The processing module 5 connects to the sensor assembly 15 which includes a multi-sensor array 2, electrodes 4a, 4b, 4c. The sensor assembly 15 connects to a pulse oximetry circuit 8 that, in turn, connects to a finger-worn pulse oximetry module 41. The sensor assembly 15 includes a male electrical connector 3 that mates with a corresponding female connector 26 on the processing module 5. The processing module 5 operates using two AA batteries 9a, 9b or equivalent rechargeable batteries.

During a measurement, the body-worn sensor 20 is worn on the patient's arm 45, and the sensor assembly 15 connects to electrodes 4a, 4b using a shielded flex cable 10. The flex cable 10 typically includes a flexible, polyimide substrate with embedded conductive traces (typically made of metal or conducting ink) that can easily and comfortably wrap around the patient's arm. In addition to the conductive traces, the cable typically has pads that optical components in the multi-sensor array can solder to. It typically features a flat, rectangular surface. The electrodes 4a, 4b adhere to the patient's skin to measure unique electrical signals. The same flex cable 10 connects to a multi-sensor array 2 that measures an optical waveform. During a measurement, both optical and electrical signals pass to an amplifier/filter circuit within the processing module 5, and from there through separate channels to the analog-to-digital converter. The serial connector 3 also includes a shielded electrical connector 18 that receives an electrical lead 13 that connects to a third electrode 4c positioned on the patient's chest. The three electrodes 4a, 4b, 4c form a proxy for an Einthoven's triangle configuration, and are used to measure a single-lead ECG. A secondary shielded electrical connector 19 connects to an acoustic sensor, not shown in figure, to measure a respiratory rate from the patient. The sensor assembly 15 further connects to a pulse oximetry circuit 8 through a separate flex cable 6. The pulse oximetry circuit connects to a pulse oximetry sensor 41 through a cable 12. A soft wristband 40 holds the cable 12 in place.

To measure optical waveforms, the multi-sensor array 2 includes three optical modules 80, 81, and 82 that collectively measure an optical waveform, or PPG, from the patient. Use of the three optical modules 80, 81, 82 increases both the signal-to-noise ratio of the optical waveform, as well as the probability that the waveform is measured from an artery, as opposed to a capillary bed. Typically an optical waveform measured from an artery yields a PTT that correlates better to blood pressure. The pulse oximetry sensor 41 measures a second optical waveform which can be processed along with the optical waveform measured with the multi-sensor array 2 to determine VTT. Each optical waveform features a time-dependent ‘pulse’ corresponding to each heartbeat that represents a volumetric change in an underlying artery caused by the propagating pressure pulse.

The electrodes 4a, 4b in the sensor assembly 15 feature metal snaps 11a, 11b to secure disposable electrode patches, not shown in figure, that attach to the patient's arm and chest. The disposable electrode patches typically feature a metal contact coated with an Ag/AgCl thin film, a solid or liquid gel component that interfaces to the patient's skin, and an adhesive component. In an alternate embodiment, these materials are embedded directly in the sensor assembly 15 (i.e. the assembly does not include metals snaps or disposable electrode patches) to form the electrode. The electrode materials generate electrical signals that, once processed, form the electrical waveform. The electrical waveform includes a sharp peak corresponding to the QRS complex. PTT is calculated for each heartbeat by measuring the time difference between the peak of the QRS complex and the foot (i.e. onset) of the optical waveform. This property is then used as described below to determine the patient's blood pressure. The process for measuring blood pressure using a multi-sensor array is described in the following co-pending patent application, the entire contents of which are incorporated herein by reference: MULTI-SENSOR ARRAY FOR MEASURING BLOOD PRESSURE (U.S. Ser. No. 12/139,219; filed Jun. 13, 2007).

The optical modules within the multi-sensor array 2 typically include an LED operating near 570 nm, a photodetector, and an amplifier. Alternatively the array can include one or more discrete LEDS and one or more discrete photodetectors. This wavelength is selected because it is particularly sensitive to volumetric changes in an underlying artery when deployed in a reflection-mode geometry, as described in the following co-pending patent application, the entire contents of which are incorporated herein by reference: SYSTEM FOR MEASURING VITAL SIGNS USING AN OPTICAL MODULE FEATURING A GREEN LIGHT SOURCE (U.S. Ser. No. 11/307,375; filed Feb. 3, 2006). 570 nm is also particularly effective at measuring optical waveforms from a wide range of skin types featuring different levels of pigmentation. Use of this wavelength is described, for example, in the following technical paper, the contents of which are incorporated herein by reference: ‘Racial Differences in Aortic Stiffness in Normotensive and Hypertensive Adults’, Journal of Hypertension. 17, 631-637, (1999). A preferred optical module is the TRS1755 manufactured by TAOS Inc. of Plano, Tex. (www.taosinc.com).

Typically, three optical modules are used in the multi-sensor array 2 to increase the effective optical field and, consequently, the probability that an underlying or proximal artery is measured. This in turn increases both the strength of the optical signal and its signal-to-noise ratio. Operating in concert, the three sensors collectively measure an optical waveform that includes photocurrent generated by each optical module. The resultant signal forms the optical waveform, and effectively represents an ‘average’ signal measured from vasculature (e.g., arteries and capillaries) underneath or proximal to the sensor 2.

The above-described system determines the patient's blood pressure using PTT, and then corrects this value for VI using algorithms described in the following patent application, the entire contents of which are incorporated herein by reference: VITAL SIGN MONITOR FOR CUFFLESSLY MEASURING BLOOD PRESSURE CORRECTED FOR VASCULAR INDEX (U.S. Ser. No. 12/138,199; filed Jun. 12, 2008). Specifically, it is well know that a patient's arteries stiffen with biological age. This property can thus be used to estimate the patient's vascular stiffness. When used with a PTT-based measurement of blood pressure, which depends strongly on vascular stiffness, biological age can therefore reduce the need for calibration and increase the accuracy of the blood pressure measurement. The accuracy of the measurement can be further improved with VI, which serves as a proxy for a ‘true’ age of the patient's vasculature: patients with elastic arteries for their age will have a VI lower than their biological age, while patients with stiff arteries for their age will have a VI greater than their biological age. The difference between VI and the patient's biological age can be compared to a pre-determined correction factor to improve the accuracy of a PTT-based blood pressure measurement.

In an alternate embodiment, the body-worn sensor system 20 can be integrated with a conventional blood pressure cuff and used to perform a blood pressure measurement called the ‘Composite Technique’, as described in the following patent application, the entire contents of which are incorporated herein by reference: VITAL SIGN MONITOR MEASURING BLOOD PRESSURE USING OPTICAL, ELECTRICAL, AND PRESSURE WAVEFORMS (U.S. Ser. No. 12/138,194; filed Jun. 12, 2008).

Referring to FIG. 2A, the body-worn sensor system 20 is designed to wrap around the arm of an average patient. The dimensions of the body-worn sensor (in inches) are as follows:

D1=2.5

D2=3.0

D3=11

D4=8

D5=5.5

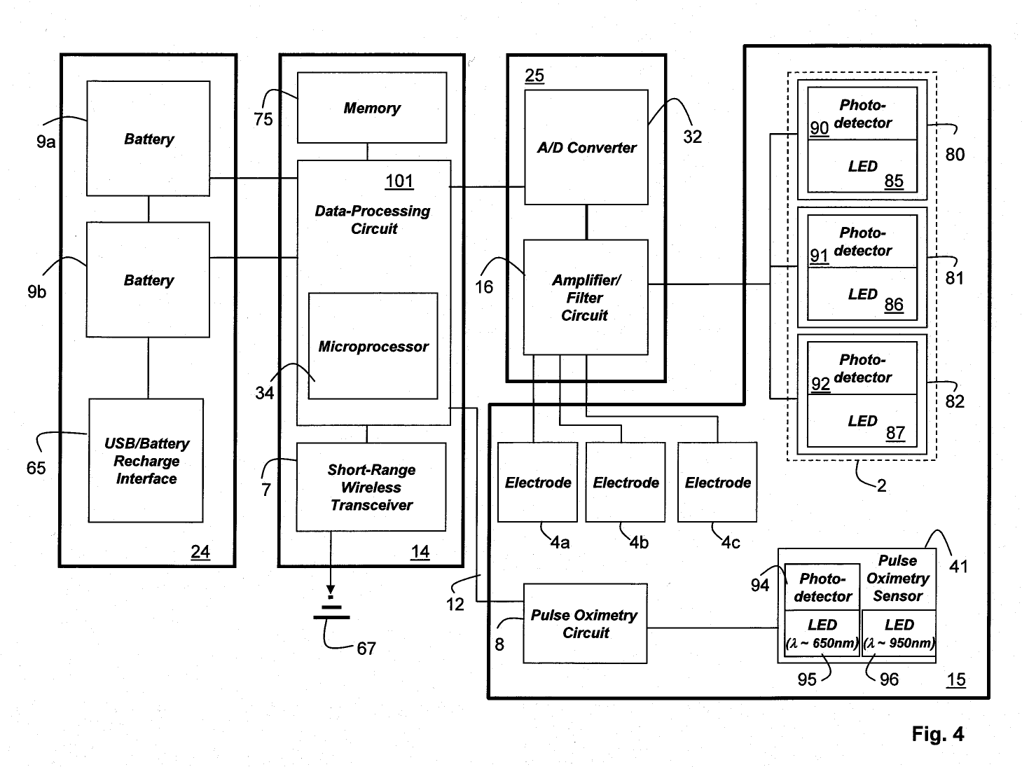

As shown in FIGS. 3A, 3B, and 4, to minimize size, the processing module 5 is constructed using three circuit boards: a main circuit board 14 and analog board 25 are disposed horizontally, and are connected by a power regulating board 24, which is disposed vertically. During a measurement, an electrical current is drawn from the AA batteries 9a, 9b through positive 27a, 28a and ground 27b, 28b battery terminals connected to the power regulating board 24. The main circuit board 14 houses the data-processing circuit 101 and microprocessor 34 and controls the sensor assembly 15. As described above, the sensor assembly includes three electrodes 4a, 4b, 4c and a multi-sensor array 2 that includes three optical modules 80, 81, and 82. Each optical module includes an LED 85, 86, 87 operating near 570 nm, and a photodetector 90, 91, 92 that detects reflected radiation at this wavelength. During operation, the main circuit board 14 receives signals from the analog board 25, which processes the optical and electrical signals directly from the sensor assembly 15. Each optical and electrical signal is amplified by an amplifier/filter circuit 16 using separate amplifier and filter circuits. This generates analog optical and electrical signals, which are is then digitized with an analog-to-digital converter 32. The analog-to-digital converter 32 is typically a separate integrated circuit (manufacturer: Texas Instruments; part number: ADS8344NB) that digitizes the waveforms at rates typically between 250-1000 Hz with 16-bit resolution. Such high resolution is required to adequately process the optical and electrical waveforms and generate an accurate PTT value. The data-processing circuit 101 is programmed with computer code that controls the body-worn sensor's various functions. The computer code runs on a high-end microprocessor 34, typically an ARM 9 processor (manufacturer: Atmel; part number: AT91SAM9261-CJ) contained in a conventional ball grid array package. Once digitized, the optical and electrical waveforms can be stored in memory 75. The pulse oximetry sensor 41 is in direct communication with the pulse oximetry circuit 8, and includes separate LEDs 95, 96 operating near, respectively, 650 nm and 950 nm, and a photodetector 94. The pulse oximetry circuit 8 determines a pulse oxygenation value from a patient, and connects directly to the data processing circuit 101. A preferred pulse oximeter module is provided by SPO Medical; part number: PulseOx 7500™.

The processing module 5 communicates using a short-range wireless transceiver 7 that transmits information through an on-board ceramic antenna 67 to a matched transceiver in a remote device. The short-range wireless transceiver can be a Bluetooth® transceiver 7, or alternatively a wireless transceiver that operates on a wireless local-area network, such as a WiFi® transceiver. The processing module can also use a USB connection 65 to communicate with external devices or recharge the AA batteries.

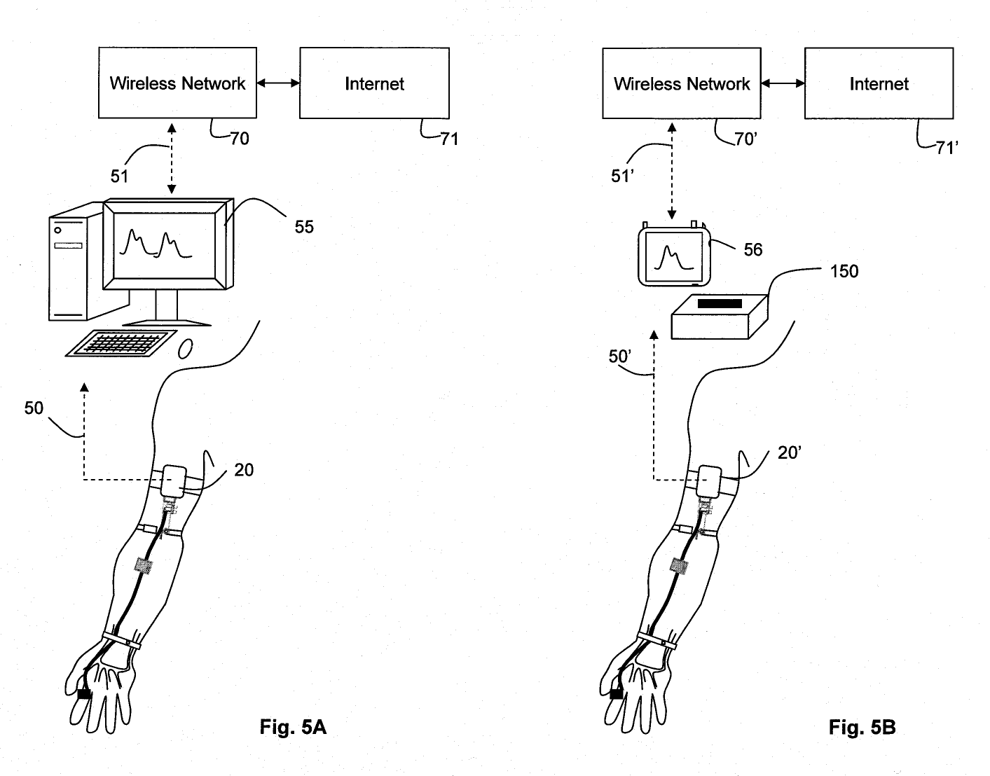

FIGS. 5A and 5B show a patient wearing the body-worn sensor system 20, 20′ in wireless communication 50, 50′ with a personal computer 55 or handheld display component 56. The personal computer 55 or handheld display component 56 is in further communication through a wireless interface 51, 51 ′ with a wireless network 70, 70′ that connects to the Internet 71, 71′. The handheld display component 56 is highly portable and can be easily removed from a docking station 150.

A number of additional solutions can be used to calculate blood pressure from PTT measured as described above. Such method are described in the following co-pending patent applications, the contents of which are incorporated herein by reference:
1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No.; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) PATCH SENSOR FOR MEASURING VITAL SIGNS (U.S. Ser. No. 11/160,957; filed Jul. 18, 2005); 10) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 11) HAND-HELD MONITOR FOR MEASURING VITAL SIGNS (U.S. Ser. No. 11/162,742; filed Sep. 21, 2005); 12) CHEST STRAP FOR MEASURING VITAL SIGNS (U.S. Ser. No. 11/306,243; filed Dec. 20, 2005); 13) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 14) BILATERAL DEVICE, SYSTEM AND METHOD FOR MONITORING VITAL SIGNS (U.S. Ser. No. 11/420,281; filed May 25, 2006); 15) SYSTEM FOR MEASURING VITAL SIGNS USING BILATERAL PULSE TRANSIT TIME (U.S. Ser. No. 11/420,652; filed May 26, 2006); 16) BLOOD PRESSURE MONITOR (U.S. Ser. No. 11/530,076; filed Sep. 8, 2006); 17) TWO-PART PATCH SENSOR FOR MONITORING VITAL SIGNS (U.S. Ser. No. 11/558,538; filed Nov. 10, 2006); and, 18) MONITOR FOR MEASURING VITAL SIGNS AND RENDERING VIDEO IMAGES (U.S. Ser. No. 11/682,177; filed Mar. 5, 2007).

Other embodiments are also within the scope of the invention. For example, the system is not limited to three optical modules. Additional optical modules could be added to further strengthen the magnitude of the optical waveform. Also, the optical modules within the multi-sensor array are not limited to the ‘linear’ form factor shown in FIG. 1. The modules, for example, may be placed in a circular configuration, may be offset from one another, or may be fashioned in a random distribution to irradiate a relatively large area of underlying skin. Such a configuration may be desirable for patients with a darker pigmented skin. In other embodiments, additional electrodes may be added to strengthen the electrical waveform.

Further embodiments are within the scope of the following claims:

What is claimed is:

1. A system for measuring a patient's blood pressure over a length of time, the system comprising a sensor assembly featuring a flexible cable configured to wrap around a portion of a patient's arm, the flexible cable having a back surface and comprising:
at least two electrodes, mounted on the back surface and positioned to contact the patient's skin to generate electrical signals when the sensor assembly wraps around a portion of the patient's arm;
an optical sensor, mounted on the back surface and comprising at least one light source and at least one photodetector, wherein the at least one light source and at least one photodetector are positioned to be adjacent to the patient's skin when the sensor assembly wraps around a portion of the patient's arm, wherein the optical sensor is configured to generate an optical signal by detecting optical radiation emitted by the at least one light source and reflected from a blood vessel underneath the patient's skin;
the system further comprising a controller configured to be worn on the patient's body, and configured to connect to the sensor assembly through a connector, the controller comprising:
i) an analog-signal processing circuit comprising a first amplifier configured to receive the electrical signals from the electrodes and generate an analog electrical waveform therefrom, and a second amplifier configured to receive the optical signal from the photodetector and generate an analog optical waveform therefrom, and further comprising an analog-to-digital converter configured to receive the analog electrical waveform and generate a digital electrical waveform therefrom, and to receive the analog optical waveform and generate a digital optical waveform therefrom; ii) a central processing circuit configured to receive the digital electrical and optical waveforms and determine a pulse transit time which is a measure of a separation in time of a first feature of the digital electrical waveform and a second feature of the digital optical waveform, and to use the pulse transit time to determine a blood pressure value for a patient; and, iii) a power-regulating circuit configured to manage power supplied to the analog-signal processing circuit and central processing circuit.

2. The system of claim 1, wherein the flexible cable comprises a rectangular cross section.

3. The system of claim 2, wherein the flexible cable comprises a polymer base.

4. The system of claim 3, wherein the flexible cable comprises a first set of metal pads for mounting the at least one light source, and a second set of metal pads for mounting the at least one photodetector.

5. The system of claim 1, wherein the flexible cable further comprises at least one connector that mates to a connector comprised by a disposable electrode.

6. The system of claim 1, wherein at least one electrode is adhered to the flexible cable with an adhesive.

7. The system of claim 1, wherein the flexible cable comprises a first connector in electrical contact with the at least two electrodes, the light source, and the photodetector, and the controller comprises a second connector configured to mate with the first connector, wherein the second connector is in electrical contact with the analog-signal processing circuit.

8. The system of claim 1, wherein the flexible cable further comprises a light source operating near 570 nm.

9. The system of claim 1, further comprising an array of light sources.

10. The system of claim 1, further comprising a short-range wireless transceiver configured to transmit information to a remote receiver.

11. A system for measuring a patient's blood pressure over a length of time, the system comprising a sensor assembly featuring a flexible cable configured to wrap around a portion of a patient's arm, the flexible cable having a flat, rectangular surface and comprising:
at least two electrodes, mounted on the flat rectangular surface and positioned to contact the patient's skin to generate electrical signals when the sensor assembly wraps around a portion of the patient's arm;
an optical sensor, mounted on the flat rectangular surface and comprising at least one light source and at least one photodetector, wherein the at least one light source and at least one photodetector are positioned to be adjacent to the patient's skin when the sensor assembly wraps around a portion of the patient's arm, wherein the optical sensor is configured to generate an optical signal by detecting optical radiation emitted by the at least one light source and reflected from a blood vessel underneath the patient's skin;
the system further comprising a controller configured to be worn on the patient's body, and configured to connect to the sensor assembly through a connector, the controller comprising:
i) an analog-signal processing circuit comprising a first amplifier configured to receive the electrical signals from the electrodes and generate an analog electrical waveform therefrom, and a second amplifier configured to receive the optical signal from the photodetector and generate an analog optical waveform therefrom, and further comprising an analog-to-digital converter configured to receive the analog electrical waveform and generate a digital electrical waveform therefrom, and to receive the analog optical waveform and generate a digital optical waveform therefrom; ii) a central processing circuit configured to receive the digital electrical and optical waveforms and determine a pulse transit time which is a measure of a separation in time of a first feature of the digital electrical waveform and a second feature of the digital optical waveform, and to use the pulse transit time to determine a blood pressure value for a patient; and, iii) a power-regulating circuit configured to manage power supplied to the analog-signal processing circuit and central processing circuit.

12. The system of claim 11, wherein the flexible cable comprises a polymer base.

13. The system of claim 12, wherein the flexible cable comprises a first set of metal pads for mounting the at least one light source, and a second set of metal pads for mounting the at least one photodetector.

14. The system of claim 11, wherein the flexible cable further comprises at least one connector that mates to a connector comprised by a disposable electrode.

15. The system of claim 11, wherein at least one electrode is adhered to the flexible cable with an adhesive.

16. The system of claim 11, wherein the flexible cable comprises a first connector in electrical contact with the at least two electrodes, the light source, and the photodetector, and the controller comprises a second connector configured to mate with the first connector, wherein the second connector is in electrical contact with the analog-signal processing circuit.

17. The system of claim 11, wherein the flexible cable further comprises a light source operating near 570 nm.

18. The system of claim 11, further comprising an array of light sources.

19. The system of claim 11, further comprising a short-range wireless transceiver configured to transmit information to a remote receiver.

20. A system for measuring a patient's blood pressure over a length of time, the system comprising a sensor assembly featuring a flexible cable configured to wrap around a portion of a patient's arm, the flexible cable having a flat, rectangular surface and comprising:
at least two electrodes, mounted on the flat rectangular surface and positioned to contact the patient's skin to generate electrical signals when the sensor assembly wraps around a portion of the patient's arm;
an optical sensor, mounted on the flat rectangular surface and comprising at least one light source and at least one photodetector, wherein the at least one light source and at least one photodetector are positioned to be adjacent to the patient's skin when the sensor assembly wraps around a portion of the patient's arm, wherein the optical sensor is configured to generate an optical signal by detecting optical radiation emitted by the at least one light source and reflected from a blood vessel underneath the patient's skin.

21. The system of claim 20, further comprising a controller configured to be worn on the patient's body, and configured to connect to the sensor assembly through a connector, the controller comprising:
i) an analog-signal processing circuit comprising a first amplifier configured to receive the electrical signals from the electrodes and generate an analog electrical waveform therefrom, and a second amplifier configured to receive the optical signal from the photodetector and generate an analog optical waveform therefrom, and further comprising an analog-to-digital converter configured to receive the analog electrical waveform and generate a digital electrical waveform therefrom, and to receive the analog optical waveform and generate a digital optical waveform therefrom; ii) a central processing circuit configured to receive the digital electrical and optical waveforms and determine a pulse transit time which is a measure of a separation in time of a first feature of the digital electrical waveform and a second feature of the digital optical waveform, and to use the pulse transit time to determine a blood pressure value for a patient; and, iii) a power-regulating circuit configured to manage power supplied to the analog-signal processing circuit and central processing circuit.
